(19) United States
(12) Patent Application Publication
(10) Pub. No.: US 2026/0088141 A1
(43) Pub. Date: Mar. 26, 2026

(54) SYSTEM AND METHOD FOR DETERMINING TESTING AND TREATMENT

(71) Applicant: Intervention Insights, Inc., Burlington, MA (US)

(72) Inventors: Clynt Taylor, Burlington, MA (US); Russell Ingersoll, Burlington, MA (US)

(73) Assignee: Intervention Insights, Inc., Burlington, MA (US)

(21) Appl. No.: 19/186,164

(22) Filed: Apr. 22, 2025

Related U.S. Application Data
(63) Continuation of application No. 17/552,839, filed on Dec. 16, 2021, now abandoned.
(60) Provisional application No. 63/126,002, filed on Dec. 16, 2020.

(51) Int. Cl. G16H 10/40; G16H 10/20; G16H 10/60; G16H 50/20; G16H 70/20

(52) U.S. Cl. CPC G16H 10/40; G16H 10/20; G16H 10/60; G16H 50/20; G16H 70/20

(57) ABSTRACT

A system and method for determining a testing and treatment protocol for a predetermined medical condition are provided. The system comprises having a processor and a non-transitory computer readable medium, a medical evidence database written on and stored to the non-transitory computer readable medium, and a processor configured to execute the computer executable instructions embodied on the non-transitory computer readable medium, and thereby execute the present method of determining a testing and treatment protocol for a predetermined medical condition including: comparing a patient profile to a predetermined set of patient characteristics defined by a plurality of patient cohorts; matching the patient profile to a patient cohort; identifying markers for evaluation and testing based on the matched patient cohort; matching the identified markers to a plurality of test order sets; matching a treatment protocol to the patient profile based on a result returned by the test order set.

### CROSS REFERENCE TO RELATED APPLICATIONS

This application is a continuation of U.S. patent application Ser. No. 17/552,839, filed on Dec. 16, 2021, and entitled System and Method for Determining Testing and Treatment, which claims the benefit of U.S. Provisional Application No. 63/126002, filed Dec. 16, 2020. Each of the foregoing are incorporated herein by reference in their entirety.

### TECHNICAL FIELD

The disclosure relates generally to a system and method for determining a testing and treatment protocol for a predetermined a medical condition.

### BACKGROUND

Over the past few decades, dramatic developments in basic science have vastly improved the understanding of disease at the molecular and or genetic level, i.e., how individual variability in the genes and proteins of human bodies contribute to cancer and other conditions such as heart disease or autoimmune disease. New methods for testing the genetic variability in a clinical setting have dramatically improved the prospect of applying precision medicine, i.e., personalized medicine, to an increasingly broader spectrum of the population.

Significant impacts of precision medicine today are seen in Oncology. Indeed, precision medicine has resulted in a shift in the treatment of certain types of cancer. Generally, cancers are largely driven by molecular errors in genes or “mutations.” These mutations are either inherited or, more commonly, are a result of an accumulation of genomic damage during a subject's life. Many new treatments have been developed to target mutations, and these “targeted therapies” have proven highly efficacious.

However, cancer is not the only disease to benefit from a growing clinical knowledge of genetics. As a consequence, genetic and molecular testing is advantageous for identifying patients who will benefit from a targeted treatment or protocol and to avoid treating those who—due to their particular genetic makeup—cannot benefit. Such precise matching of treatment to molecular test results is likely beneficial to the care for and resultant outcome provided to patients. As such, there exists a need for a solution that is configured to keep up with the daily growth in clinical knowledge and to apply this knowledge consistently for all patients before physicians make diagnostic testing and treatment decisions.

### SUMMARY

A system and method for determining testing a treatment protocol for a predetermined medical condition are provided. The system comprises a computing device having a processor and a non-transitory computer readable medium, a medical evidence database written on and stored to the non-transitory computer readable medium, and a processor configured to execute the computer executable instructions embodied on the non-transitory computer readable medium, and thereby execute the present method.

The medical evidence database is populated from designated sources via an automated information gathering device, wherein the automated information gathering device is programmed to retrieve information from a predefined medical research source. The automated information gathering device returns the targeted medical research information to the computer readable medium, wherein the targeted medical research information is evaluated and organized according to a predetermined set of evaluation criteria, into a plurality of patient cohorts, wherein each of the plurality of patient cohorts comprises a predetermined set of patient characteristics.

The present method for determining a testing and treatment protocol for a predetermined medical condition comprises the following steps: comparing a patient profile, obtained via a patient questionnaire, interview or the like, to a predetermined set of patient characteristics defined by a plurality of patient cohorts; matching the patient profile to a patient cohort; identifying markers for medical evaluation and testing based on the matched patient cohort; matching the identified markers for medical evaluation and testing to a plurality of test order sets; matching a treatment protocol to the patient profile based on a result returned by the test order set.

The above features and advantages, and other features and advantages, of the present teachings are readily apparent from the following detailed description of some of the best modes and other embodiments for carrying out the present teachings, as defined in the appended claims, when taken in connection with the accompanying drawings.

### BRIEF DESCRIPTION OF THE DRAWINGS

The operation of the invention may be better understood by reference to the detailed description taken in connection with the following illustrations, wherein:

FIG. 1 is a schematic block diagram of an example system for determining a testing and treatment protocol for a predetermined medical condition;

FIG. 2 is an example flow chart detailing the steps of the present method;

FIG. 3 is an example flow chart detailing the sub-steps of matching the identified markers for medical evaluation and testing to a plurality of test order sets;

FIG. 4A illustrates a diagram of an example rule set used within the medical evidence database;

FIG. 4B illustrates a table showing the processing of lab test options for determining potential test order sets and a minimum viable test order set;

FIG. 4C illustrates a table showing the processing of test order sets through health plan policies; and

FIG. 4D illustrates a system diagram of the system and method determining a testing and treatment protocol for a predetermined a medical condition.

### DETAILED DESCRIPTION

While the present disclosure may be described with respect to specific applications or industries, those skilled in the art will recognize the broader applicability of the disclosure. The terms “a”, “an”, “the”, “at least one”, and “one or more” are used interchangeably to indicate that at least one of the items is present. A plurality of such items may be present unless the context clearly indicates otherwise. All numerical values of parameters (e.g., of quantities or conditions) in this specification, unless otherwise indicated expressly or clearly in view of the context, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, a disclosure of a range is to be understood as specifically disclosing all values and further divided ranges within the range.

The terms “comprising”, “including”, and “having” are inclusive and therefore specify the presence of stated features, steps, operations, elements, or components, but do not preclude the presence or addition of one or more other features, steps, operations, elements, or components. Orders of steps, processes, and operations may be altered when possible, and additional or alternative steps may be employed. As used in this specification, the term “or” includes any one and all combinations of the associated listed items. The term “any of” is understood to include any possible combination of referenced items, including “any one of” the referenced items. The term “any of” is understood to include any possible combination of referenced claims of the appended claims, including “any one of” the referenced claims.

Features shown in one figure may be combined with, substituted for, or modified by, features shown in any of the figures. Unless stated otherwise, no features, elements, or limitations are mutually exclusive of any other features, elements, or limitations. Furthermore, no features, elements, or limitations are absolutely required for operation. Any specific configurations shown in the figures are illustrative only and the specific configurations shown are not limiting of the claims or the description.

For consistency and convenience, directional adjectives are employed throughout this detailed description corresponding to the illustrated embodiments. Those having ordinary skill in the art will recognize that terms such as “above”, “below”, “upward”, “downward”, “top”, “bottom”, etc., may be used descriptively relative to the figures, without representing limitations on the scope of the invention, as defined by the claims. Any numerical designations, such as “first” or “second” are illustrative only and are not intended to limit the scope of the disclosure in any way.

The term “longitudinal”, as used throughout this detailed description and in the claims, refers to a direction extending a length of a component. In some cases, a component may be identified with a longitudinal axis as well as a forward and rearward longitudinal direction along that axis. The longitudinal direction or axis may also be referred to as an anterior-posterior direction or axis.

The term “transverse”, as used throughout this detailed description and in the claims, refers to a direction extending a width of a component. The transverse direction or axis may also be referred to as a lateral direction or axis or a mediolateral direction or axis.

The term “vertical”, as used throughout this detailed description and in the claims, refers to a direction generally perpendicular to both the lateral and longitudinal directions.

In addition, the term “proximal” refers to a direction that is nearer a center of a component. Likewise, the term “distal” refers to a relative position that is further away from a center of the component. Thus, the terms proximal and distal may be understood to provide generally opposing terms to describe relative spatial positions.

Referring to the drawings, wherein like reference numerals refer to like components throughout the several views, a system 10 and method 100 for determining a testing and treatment protocol for a predetermined medical condition are provided. While the system and method disclosed herein for determining a testing and treatment protocol for a predetermined medical condition is generally described as used to diagnose and determine treatments or therapies for cancer patients, it will be appreciated that the systems and methods described herein may be used in conjunction with testing and treatment of any variety of predetermined medical conditions.

In a general sense, the testing and treatment system 10 and method 100 are configured to analyze patient characteristics, determine an appropriate grouping or patient cohort 12 for a patient 50, determine appropriate markers 20 to be tested based on the patient cohort 12, determine an efficient marker testing plan, and determine potential therapies or treatment protocols 22 for the patient 50 based on test results.

More particularly, the system 10 comprises a non-transitory computer readable medium 16 that stores a set of computer executable instructions 100; an evidence database 18 written on and stored to the non-transitory computer readable medium 16, and at least one processor 14 configured to execute the computer executable instructions 100 embodied in the non-transitory computer readable medium16, such that the non-transitory computer readable medium 16 is configured to instruct the processor 14 to execute the present method 100 for determining a testing and treatment protocol 22 for a predetermined medical condition. The method 100 for determining a testing and treatment protocol for a predetermined medical condition comprises the following steps: comparing a patient profile, obtained via a patient questionnaire, interview or the like, to a predetermined set of patient characteristics defined by a plurality of patient cohorts 104; matching the patient profile to a patient cohort 105; identifying markers for medical evaluation and testing based on the matched patient cohort 106; and matching the identified markers for medical evaluation and testing to a plurality of test order sets 107; and matching a treatment protocol to the patient profile based on a result returned by the test order set 113.

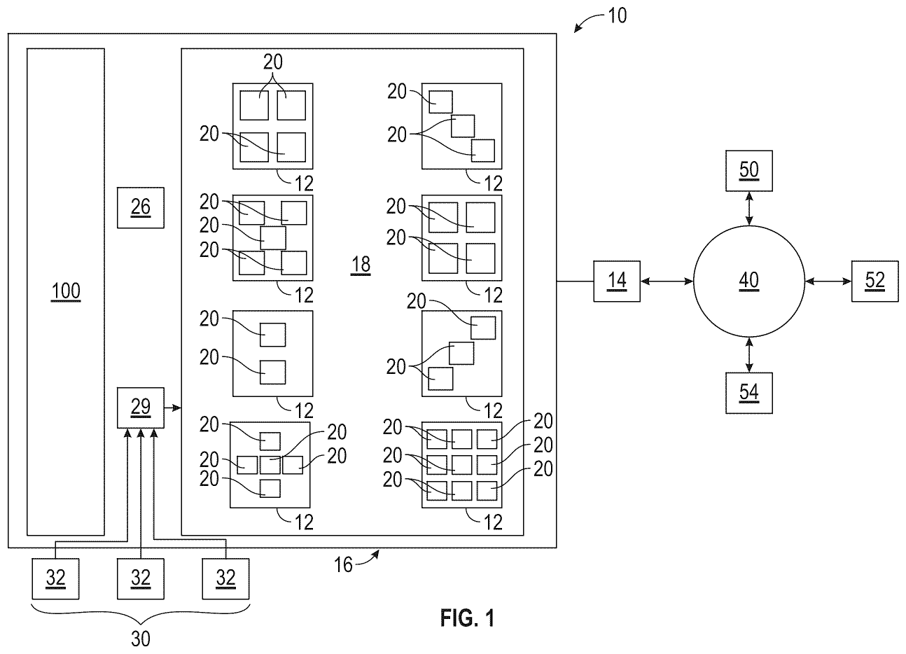

Referring to FIG. 1, the system 10 for determining a testing and treatment protocol for a predetermined medical condition is provided. The system 10 may be deployed on any one of a number of computing devices, including, without limitation, a computer workstation, a desktop, notebook, laptop, a handheld computer, a mobile phone, a tablet, or some other computing device.

The system 10 may include a non-transitory computer readable medium 16. The term non-transitory computer readable medium includes any medium that participates in providing data (e.g., instructions), which may be read by a computer. Such a medium may take many forms, including, but not limited to, non-volatile media, volatile media, etc. Non-volatile media include, for example, optical or magnetic disks and other persistent memory. Volatile media include dynamic random-access memory (DRAM), which typically constitutes a main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read, as well as networked versions of the same. The non-transitory computer readable medium 16 stores or has written or embodied thereon a set of computer executable instructions that comprise the present method 100 for determining a testing and treatment protocol 22 for a predetermined medical condition.

A user interface module 26 may also be written on or embodied in the non-transitory computer readable medium 16. The user interface module 26 may be operative to implement a graphical user interface that can be stored in a mass storage device as executable software codes that are executed by the one or more computing devices. This and other modules can include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

The system 10 further includes a medical evidence database 18 written on and stored to the non-transitory computer readable medium 16. Databases or data stores described herein may include various kinds of mechanisms for storing, accessing, and retrieving various kinds of data, including a hierarchical database, a set of files in a file system, an application database in a proprietary format, a relational database management system (RDBMS), a non-relational database management system, a look-up table, etc. Each such database or data store is generally included within a computing device employing a computer operating system and may be accessed via a network 40 in any one or more of a variety of manners.

The medical evidence database 18 is configured to store a compilation of targeted medical research information 30. The evidence database 18 is generally configured to store targeted medical research information 30, such as clinical evidence compiled from numerous sources. The medical evidence database 18 may be compiled or populated from designated sources 32 via an automated information gathering device 29 programmed to retrieve information from the predefined medical research source 32 and return the targeted medical research information 30 to populate the medical evidence database 18 written on the non-transitory computer readable medium 16.

The medical evidence database 18 may be populated in part by automated information gathering device 29 such as content crawlers. The content crawlers may comprise internet bots that are configured to seek out targeted information and retrieve the information to be organized and processed. The content crawlers may be specifically configured to seek out content from designated sources 32. For example, each content crawler may be programed or configured to seek out and retrieve information from a specific medical journal, library, study, or other predetermined or preprogrammed source 32. The content crawler may further be programmed or configured to retrieve relevant clinical data from known tests and studies. The clinical data may include data related to details of a study, such as details of a study group, study size, molecular and/or genetic components of the group, controls in place during the clinical study or test, study outcomes, responsiveness of study participants, reproducibility of results, durability of response, magnitude of response over the pool of participants, and other details and information related to the clinical study and outcomes.

The targeted medical research information 30 may be evaluated according to a predetermined set of evaluation criteria before it is entered into the evidence database 18. The targeted medical research information 30 may also be organized according to the predetermined set of organization criteria and divided or separated into a plurality of patient cohorts 12, wherein each of the plurality of patient cohorts 12 comprises a predetermined set of patient characteristics. Said another way, the targeted medical research information 30 may specifically be broken down and organized into rule sets that provide an association between a set of patient characteristics, genetic/molecular markers 20 associated with the patient characteristics, as well as an association between a genetic/molecular marker 20 and a treatment protocol 22.

The targeted medical research information 30 may be evaluated and organized into the plurality of patient cohorts 12 in both an automated manner by computers and text readable programs that are programmed to evaluate and organize such data and/or in a manual manner by humans who review and evaluate the clinical information returned by the automated information gathering device(s) 29. The manual evaluation and organizing of the targeted medical research information 30 may be performed by experts that are trained to evaluate the clinical data and input the data into the medical evidence database 18. The targeted medical research information 30 may be tagged during the evaluation process and organized based on tagged parts to identify key criteria from the clinical evidence to assist in both determinations of testing and determinations of treatment.

As used and described herein, a patient cohort 12 may comprise a set of patient characteristics that are common to patients that are defined in the targeted medical research information 30 and include a given genetic/molecular marker or markers 20. The medical evidence database 18 may be searchable by a plurality of rule sets, wherein each rule set associates a patient cohort 12 (which comprises one or more patient characteristics) with a marker 20 (which may comprise one or more genetic characteristics from one or more genes) to be tested and a potential therapy or treatment 22 (which may comprise one or more treatment modalities) to be used if the marker 20 is found.

The medical evidence database 18 may define patient cohorts 12 broadly or more narrowly depending on the number of characteristics described by the then current clinical evidence and associated with an impact on treatment outcome. The patient cohort 12 may be very broad if it only includes a low number of characteristics or may be more specific if it includes a larger number of characteristics. The broader the patient cohort 12, the greater the number of clinical characteristics (including markers 20) that may be associated with the cohort 12. For example, if a patient 50 is in a broad cohort 12 that only includes one or two characteristics, then the system 10 may recommend a testing or treatment direction that is associated with clinical evidence that only describes the same limited patient characteristics. By contrast, if a patient 50 is in a narrower cohort 12, one that includes a greater number of characteristics as described in the clinical evidence, then the combination of those characteristics (that patient cohort 12) may yield different testing or treatment 22. In either case, it will be appreciated that the set of markers 20 to be tested and subsequent treatment options 22 to be considered will be determined based on characteristics of an individual patient 50 and a patient cohort 12 set forth in the rule sets.

It will also be appreciated that the rule sets and associations between patient cohorts 12, genetic/molecular markers 20, and treatments 22 may be constantly changing as new evidence is received into the evidence database 18. Specifically, new evidence from new clinical studies may create entirely new patient cohorts 12, may alter existing patient cohorts 12, may add to or subtract from the genetic/molecular markers 20 associated with a patient cohort 12 and may add to or subtract from treatments 22 that are recommended for a patient 50 within a given patient cohort 12.

The system 10, may further comprise at least one a processor 14 configured to execute the computer executable instructions 100 embodied on the non-transitory computer readable medium 16. Computer-executable instructions may be compiled or interpreted from computer programs, software code, or algorithms created using a variety of programming languages and/or technologies, including, without limitation, and either alone or in combination, Java™, C, C++, Visual Basic, Java Script, Perl, html, etc. In general, a processor 14 (e.g., a microprocessor) receives instructions, e.g., from a memory, a computer-readable medium, etc. 16, and executes these instructions, thereby performing one or more processes, including one or more of the processes described within the present method 100. Such instructions and other data may be stored and transmitted using a variety of computer-readable media 16. It is appreciated that software modules can be callable from other modules or from themselves, and/or can be invoked in response to detected events or interrupts. The modules, computer executable instructions, and/or computing device functionality described herein are preferably implemented as software modules, but can be represented in hardware or firmware. Generally, the modules, computer executable instructions, and/or computing device functionality described herein refer to logical modules that can be combined with other modules or divided into sub-modules despite their physical organization or storage.

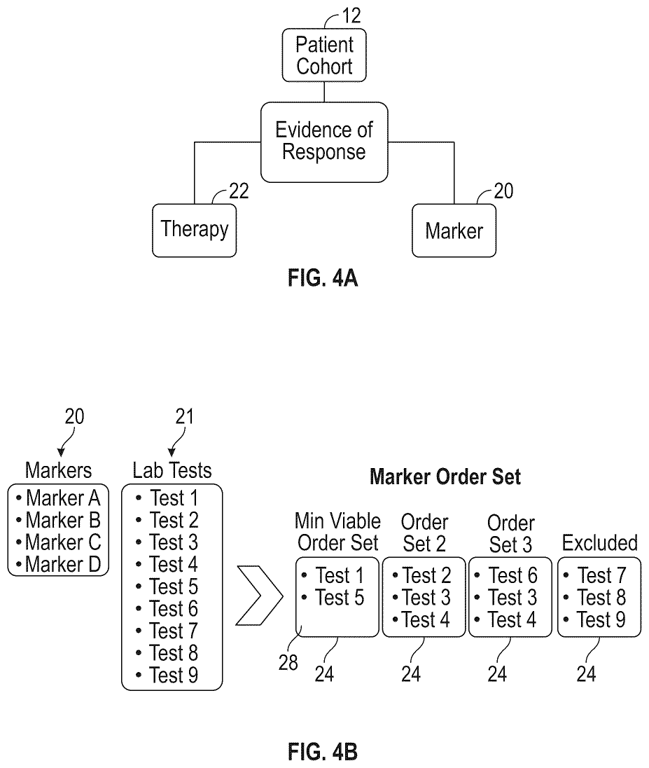

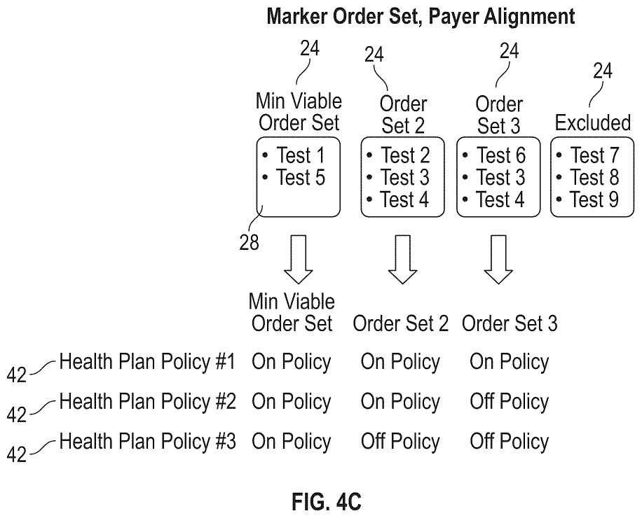

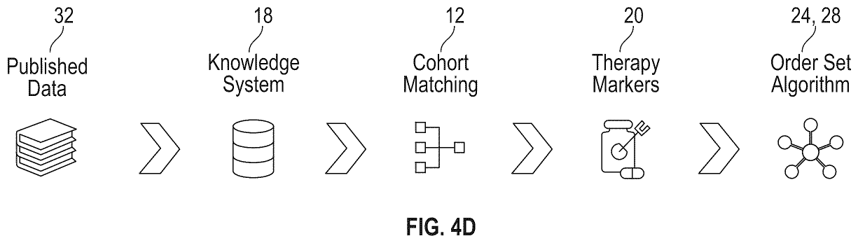

Referring to FIGS. 1 and 4D, example system diagrams are generally provided. The system 10 may populate the evidence database 18 using content crawlers that seek out available medical research and clinical information 30, including reports on cancer treatment outcomes, clinical studies, and the like. The system 10 may utilize algorithms as well as manual review to organize and tag the medical research and clinical information 30. The system 10 may define patient cohorts 12 and associate a genetic/molecular marker 20 and a therapy or treatment 22 with a patient cohort 12. In use, a patient 50 may be matched with a patient cohort 12 through a cohort matching process. The cohort matching process may comprise a conditional questionnaire that includes targeted questions to determine patient characteristics and develop a patient profile. Questions in the conditional questionnaire may be dependent on prior answers, and answers may be entered into the system 10 to develop a patient profile that is matched a patient cohort 12 for a patient 50 in real time. Once the patient cohort 12 is determined, the appropriate list of markers 20 and therapies or treatment protocols 22 may be determined. The system 10 may then identify order sets of lab tests 21 and optimize the lab testing order sets 24 based on a predetermined set of test set criteria or optimization parameters.

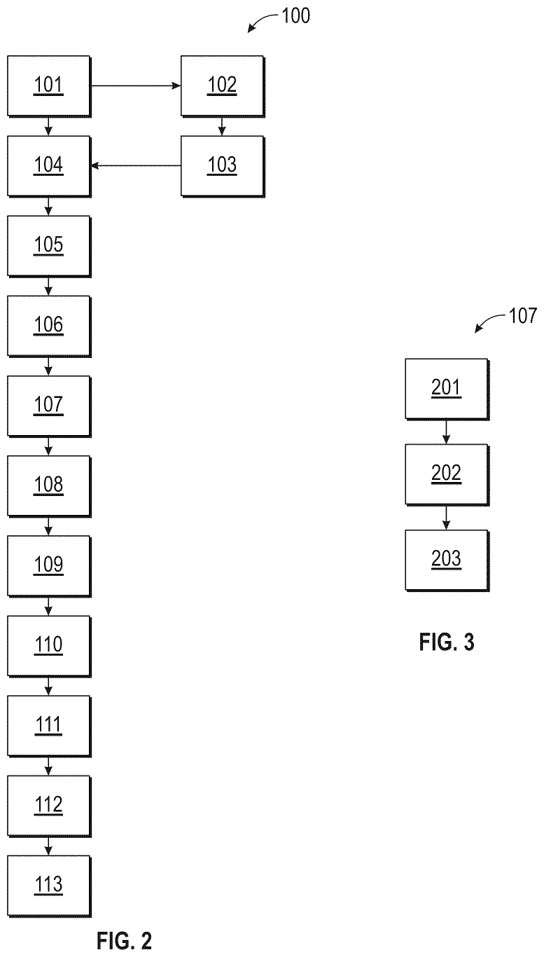

As detailed herein, the at least one a processor 14 is configured to execute the computer executable instructions embodied in the non-transitory computer readable medium 16, such that the non-transitory computer readable medium 16 is configured to instruct the processor 14 to execute the present method 100. The present method for determining testing a treatment protocol for a predetermined medical condition is detailed further in FIGS. 2-3 and 4A-4D and comprises several steps 101-113 and sub-steps 201-203.

Referring to FIG. 2 and FIG. 4A, at step 101 the medical evidence database 18 is populated with a data set of medical research information 30. Populating the medical evidence database 18 may further comprise evaluating and organizing the data set targeted medical research information 30 into a plurality of patient cohorts 12 according to a predetermined set of organization criteria, wherein each of the plurality of patient cohorts 12 comprises a predetermined set of patient characteristics.

The medical evidence database 18 may be populated in part by automated information gathering device 29 such as content crawlers. The content crawlers may be specifically configured to seek out content from designated sources. For example, each content crawler may be programed or configured to seek out and retrieve information from a specific medical journal, library, study, or other predetermined or preprogrammed source 32. The content crawler may further be programmed or configured to retrieve relevant clinical data from known tests and studies. The clinical data may include data related to details of a study, such as details of a study group, study size, molecular and/or genetic components of the group, controls in place during the clinical study or test, study outcomes, responsiveness of study participants, reproducibility of results, durability of response, magnitude of response over the pool of participants, and other details and information related to the clinical study and outcomes.

The targeted medical research information 30 may be evaluated according to a predetermined set of evaluation criteria before it is entered into the evidence database 18. The targeted medical research information 30 may also be organized according to the predetermined set of organization criteria, and divided or separated into a plurality of patient cohorts 12, wherein each of the plurality of patient cohorts 12 comprises a predetermined set of patient characteristics. Said another way, the targeted medical research information 30 may specifically be broken down and organized into rules that provide an association between a set of patient characteristics and genetic/molecular markers 20 associated with the patient characteristics, as well as an association between a genetic/molecular marker 20 and a treatment protocol 22.

As used and described herein, a patient cohort 12 may comprise a set of patient characteristics that are common to patients that are defined in the targeted medical research information 30 and include a given genetic/molecular marker or markers 20. The medical evidence database 18 may be searchable by a plurality of rule sets, wherein each rule set associates a patient cohort 12 (which comprises one or more patient characteristics) with a marker 20 (which may comprise one or more genetic characteristics from one or more genes) to be tested and a potential therapy or treatment 22 (which may comprise one or more treatment modalities) to be used if the marker 20 is found.

At step 102, the system 10 via the processor 14 and a computer network 40, may transmit a request to a patient 50. The request may comprise a questionnaire or a series of questions related to the predetermined medical condition, for which the patient 50 seeks testing and treatment. In one example, the request may comprise a conditional questionnaire administered by a treating clinician or someone under the clinician or physician's control. The questionnaire may include questions targeted to specific characteristics of the patient 50 and/or predetermined medical condition, for which the patient 50 seeks testing and treatment. Answers to each question yield further definition of the patient profile. Based on the answers to each question, a subsequent question may be generated to determine further relevant characteristics or provide additional information on given characteristics.

Once the request or questionnaire is complete, at step 103, the system 10, via the processor 14 and a computer network 40, receives a response to the request from the patient 50 and processes and stores the received response on the non-transitory computer readable medium 16 as a patient profile. The answers to the questionnaire or response to the request define the patient profile, with the specificity of the patient profile depending on the depth of questions asked and answers provided. Examples of characteristics that may be evaluated in the survey, without limitation, may include the type or location of cancer, stage of cancer, patient histology, prior treatments, age, gender, race, lifestyle activities (past and current), prior testing and results of those prior tests, familial genomic information, as well as other types of patient characteristics. The patient profile developed on the basis of the responses to the request or questionnaire may be entered into the database as the information is received and the patient profile may then be determined in real time, based on the responses.

At step 104, the system 10 compares the patient profile to a predetermined set of patient characteristics defined by each of the plurality of cohorts 12.

At step 105, the patient 50 is categorized in at least one of the plurality of patient cohorts 12 based on the patient profile.

At step 106, the system 10 identifies markers 20 for medical evaluation and testing based on the patient cohort 12. FIG. 4A illustrates a diagram of clinical evidence 18 that has been organized into a rule set. As shown, the ruleset comprises an association between the patient cohort 12, genetic/molecular markers 20 that may be associated to the set of characteristics, and a treatment protocol or therapy 22 associated with the genetic/molecular marker 20. The evidence database 18 may include a plurality of rules sets that each include a unique marker 20 and treatment 22 combination. For example, the database 18 may include a plurality of rule sets that all relate to the same or similar sets of patient characteristics, but each include a different marker 20 to be tested and/or a different treatment 22 to be proposed if the marker 20 is found.

At step 107, the system 10 matches the identified markers for medical evaluation and testing to a plurality of test order sets 24. Said another way, once the patient cohort 12 is determined, and the associated genetic/molecular markers 20 associated with that cohort 12 have been identified, lab testing options to test for the identified markers 20 may be determined. The evidence database 18 may generally include or have access to information on third-party lab test packages. The information may include details of what lab tests 21 are available, what markers 20 are tested for by each available test, and what molecular alterations for the marker 20 are detected by each available test 21. The testing options returned may be optimized to provide the most efficient and optimal package of lab tests 24.

As shown in FIG. 4B, the system 10 may cross-reference or compare the list of identified markers 20 for a patient 50 with all available and relevant lab tests 21 that test for at least one of the identified markers 20. The comparison may yield one or more sets of tests, referred to herein as test order sets 24. Each test order set 24 may comprise a list of lab tests 21 that, in total, are capable of testing for each identified marker 20 in the cohort 12 associated with the matched patient profile, keeping in mind that marker 20 means any detectable genetic event of a marker 20 that is associated in the clinical evidence 30 to a treatment modality 22 for a patient cohort 12. It will be appreciated that many lab tests 21 or lab testing packages will test for more than one marker 20, and, therefore, may potentially test for more than one marker 20 on the list of identified markers 20.

The system 10 may generate numerous order sets 24 and may review the order sets 24 to organize and reduce the same. In particular, the order sets 24 may be reduced to eliminate redundant lab tests that are not needed while still covering the complete list of markers 20. Once reduced, the most efficient or minimum viable order set 28 may be determined (FIGS. 4B and 4C). The minimum viable order set 28 may comprise the order set that requires the fewest number of lab tests 21 to test for the full set of markers 20. Order sets 24 other than the minimum viable order set 28 may be determined in order to provide testing options, as discussed further below with reference to FIG. 3.

As shown in FIG. 3, step 107 may further comprise sub-steps 201-203. At step 201, a testing laboratory 52 (FIG. 1) is selected. At step 202, a plurality of order sets 24, provided by the selected testing lab 52 is determined. In this way, the system 10 matches the identified markers 20 for medical evaluation and testing with the order sets 24 provided by the selected testing laboratory 52.

At step 203, again, the system 10 may generate numerous order sets 24 and may review the order sets 24 to organize, reduce, and rank the plurality of test order sets 24 according to the predetermined set of test set criteria. In particular, the order sets 24 may be reduced to eliminate redundant lab tests that are not needed while still covering the complete list of markers 20.

As further detailed with respect to FIG. 4C, the test order set 24 may be further categorized and ranked according to a predetermined set of test set criteria. The predetermined set of test set criteria may comprise, for example, an amount of medical tests 21 contained in the respective test order set 24, a cost of the test order set 24 to a patient 50, reimbursement liability based on laboratory supplied Clinical Procedural Codes (CPT codes), a status of the testing laboratory 52 in a predetermined patient insurance network or health care plan 42, medical provider 54 preferences, amongst other factors.

Said another way, the order sets 24 may be ranked based on how efficiently the order set 24 tests for the identified markers 20, including how many unnecessary tests 21 are included, how well the order set 24 minimizes the number of test products 21 to be used, and how well the order set 24 incorporates, as needed, reimbursement liability based on laboratory supplied Clinical Procedural Codes (CPT codes) and reduced rates based on patient healthcare plans 42 and other ranking criteria.

With respect to the patient healthcare plan 42, the system 10 may consider various factors of the healthcare plans 42 to determine each order set's 24 compliance with plan preferences. The various factors may include which labs 52 are in network, cost comparisons for preferred labs, non-preferred labs, and out-of-network labs, CPT codes including non-reimbursable CPT codes and CPT code stacks, and other similar healthcare plan 42 factors. As illustrated in the FIG. 4C, the lab tests 21 in each order set 24 may be compared with one or more healthcare plans 42 to determine if lab tests 21 are on policy or if the lab tests 21 in each order set 24 comply with the factors identified above. Based on this comparison, the optimal order set 24 of tests may be determined by the system 10.

Referring back to FIG. 2, at step 108, a test order set 24 is selected. At step 109, the system 10 transmits, via the computer network 40, the test order set 24 selection and an authorization request to a medical provider 54. At step 109, the system 10 receives, via the computer network 40, a medical provider 54 authorization in response to the authorization request. At step 110, the system 10, transmits, via the computer network 40, the test order set 24 selection and medical provider 54 authorization to the selected testing laboratory 52 and the selected testing laboratory 52 completes the grouping of lab tests 21 defined by the selected order set 24.

At step 111, the system 10 receives, via the computer network 40, a set of results from the selected testing laboratory 52 corresponding to the test order set 24. At step 112, the system 10 transmits the set of results to the medical provider 54 for evaluation. At step 113, the patient profile is matched to an available treatment protocol 22 or medical therapy based on the set of results from the selected testing laboratory 52 corresponding to the test order set 24.

With regard to the media, processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claimed invention.

The detailed description and the drawings or figures are supportive and descriptive of the present teachings, but the scope of the present teachings is defined solely by the claims. While some of the best modes and other embodiments for carrying out the present teachings have been described in detail, various alternative designs and embodiments exist for practicing the present teachings defined in the appended claims.

1. A method of treatment for a subject in need thereof comprising the steps of:
comparing a patient profile of the subject to a predetermined set of patient characteristics defined by a plurality of predetermined patient cohorts, the plurality of predetermined patient cohorts defined by a medical evidence database that is configured to store a compilation of targeted medical research information organized and evaluated according to a predetermined set of evaluation and organization criteria;
matching the patient profile to at least one of the plurality of predetermined patient cohorts; identifying markers for medical evaluation and testing based on the predetermined patient cohort;
matching the identified markers for medical evaluation and testing to a plurality of test order sets, and plurality of test order sets wherein matching the identified markers further comprises:
selecting a testing laboratory;
determining a plurality of test order sets, provided by the selected testing lab, that matches the identified markers for medical evaluation and testing;
categorizing the plurality of test order sets according to a predetermined set of test set criteria, wherein the predetermined set of test set criteria comprises an amount of medical tests
contained in the respective test order set, a cost of the test order set to a patient, and a status of the testing laboratory in a predetermined patient insurance network;
ranking the plurality of test order sets according to the predetermined set of test set criteria;
selecting a test order set;
transmitting, via a computer network, the test order set selection and an authorization request to a medical provider; and
receiving, via the computer network, a medical provider authorization in response to the authorization request;
transmitting, via the computer network, the test order set selection and medical provider authorization to the selected testing laboratory;
receiving, via the computer network, a test result from the selected testing laboratory based on the test order set and transmitting the result to the medical provider for evaluation; matching a treatment protocol to the patient profile based on the test result; and
administering the one or more treatment protocols to the subject.

2. The method of claim 1 wherein matching the identified markers for medical evaluation and testing to a plurality of test order sets further comprises grouping a set of testing products matched with the identified markers for medical evaluation and testing into the plurality of order sets.

3-7. (canceled)

8. The method of claim further comprising:
transmitting, via a computer network, an information request to a patient, wherein the information request comprises a series of questions related to the predetermined medical condition; and
receiving answers to the information request from the patient and building the patient profile based on the answers to the information request.

9. The method of claim 1 wherein:
the medical evidence database is stored on a computer readable medium.

10. The method of claim 9 further comprising building the evidence database, wherein building the evidence database further comprises:
obtaining compilation of targeted medical research information from a predefined medical research source via an automated information gathering device programmed to retrieve the targeted medical research information from the predefined medical research source;
returning the targeted medical research information from the automated information gathering device to a computer readable medium, wherein the computer readable medium is a memory;
populating the medical evidence database with the obtained and returned targeted medical research information, wherein populating the medical evidence database further comprises:
evaluating the returned targeted medical research information according to a predetermined set of evaluation and organization criteria; and
organizing the returned targeted medical research information according to the predetermined set of evaluation and organization criteria, into a plurality of patient cohorts, wherein each of the plurality of patient cohorts comprises a predetermined set of patient characteristics.

11-20. (canceled)
